(19) United States
(12) Patent Application Publication
(10) Pub. No.: US 2017/0215707 A1
(43) Pub. Date: Aug. 3, 2017

(54) SHEATH FOR AN IMAGING DEVICE

(71) Applicant: Boston Scientific Scimed, Inc., Maple Grove, MN (US)

(72) Inventors: Timothy P. HARRAH, Cambridge, MA (US); Christopher L. OSKIN, Grafton, MA (US); Derrick LENZ, Pompton Plains, NJ (US); Arpita BANERJEE, Bangalore (IN); Pavan MISRA, Bangalore (IN); Abhijit TAKALE, Pune (IN); Sandesh GAVADE, Bangalore (IN)

(73) Assignee: Boston Scientific Scimed, Inc.

(21) Appl. No.: 15/416,420

(22) Filed: Jan. 26, 2017

Related U.S. Application Data
(60) Provisional application No. 62/288,686, filed on Jan. 29, 2016.

(51) Int. Cl. A61B 1/00; A61B 1/303

(52) U.S. Cl. CPC A61B 1/00135; A61B 1/00082; A61B 1/0014; A61B 1/00154; A61B 1/303

(57) ABSTRACT

A sheath for an imaging device may include a shaft including a proximal end, a distal end, and a lumen extending from the proximal end to the distal end; a holder coupled to the proximal end of the shaft; a retainer positioned in the lumen of the shaft, wherein the retainer includes an expanded configuration and a collapsed configuration; and a light positioned on the distal end of the shaft.

### CROSS-REFERENCE TO RELATED APPLICATION

This application claims the benefit of priority under 35 U.S.C. §119 to U.S. Provisional Patent Application No. 62/288,686, filed Jan. 29, 2016, the entirety of which is incorporated herein by reference.

### TECHNICAL FIELD

The present disclosure relates generally to medical devices for providing an access pathway through a patient's urinary tract.

### BACKGROUND

A ureteroscopy is an examination of the ureter or kidney of a patient using a ureteroscope. Ureteroscopes typically include a long, thin, flexible portion that can be inserted through the patient's urethra, bladder, and ureteral orifice connecting the bladder and the ureter. Ureteroscopes typically further include a camera and lighting system to adequately visualize the scope's pathway and the working area within the ureter or kidney. Often, a ureteroscopy procedure involves treating a stone that has lodged in the patient's mid to upper ureter or within a calyx of the kidney.

Currently, before a ureteroscope can be inserted into a patient's ureter, a separate device called a cystoscope is required to pass through the urethra, examine the bladder, and find the ureteral orifice. An examination of the bladder is called a cystoscopy. The cystoscope includes a separate camera and lighting system, is often shorter than a ureteroscope, and is typically more rigid than a ureteroscope. Once the ureteral orifice has been located by the cystoscope, a guidewire is inserted into the ureter. The cystoscope is then removed from the patient, leaving the guidewire in place. An access sheath is often then inserted over the guidewire through the urethra, bladder, and into the ureter. The guidewire is removed and the ureteroscope can then be inserted through the access sheath and into the ureter.

The use of two different scopes and accompanying equipment during a ureteroscopy is expensive. Furthermore, the additional steps required to insert and remove the cystoscope from the patient and subsequently insert the ureteroscope through the urethra, bladder, and ureter increases the length of the procedure.

### SUMMARY

Examples of the present disclosure relate to, among other things, a sheath for an imaging device and methods for using the sheath with the imaging device. Each of the examples disclosed herein may include one or more of the features described in connection with any of the other disclosed examples.

A sheath for an imaging device may include an elongated shaft including a proximal end, a distal end, and a lumen extending from the proximal end to the distal end; a holder coupled to the proximal end of the shaft; a retainer positioned in the lumen of the shaft, wherein the retainer includes an expanded configuration and a collapsed configuration; and a light positioned on the distal end of the shaft.

A sheath according to any example described herein may include one or more of the following features: the retainer may include a plurality of expandable components, and each of the expandable components may be configured to be transitioned between an expanded configuration and a collapsed configuration; each of the expandable components may include a balloon; the plurality of expandable components may arranged around a circumference of an interior wall of the lumen, with a circumferential space between adjacent expandable components, so that a cross-section of the shaft perpendicular to a longitudinal axis of the shaft intersects each of the plurality of expandable components; the light may be positioned on a distal-facing surface of the distal end of the shaft; the holder may include a light switch configured to turn the light on and off; and the sheath may further comprise an inlet protruding radially outward from the shaft, the inlet being fluidly coupled to the retainer.

In another example, a sheath for an imaging device may include an elongated shaft including a proximal end, a distal end, and a lumen extending from the proximal end to the distal end; a plurality of expandable components positioned in the lumen, where each of the expandable components is configured to be transitioned between an expanded configuration and a collapsed configuration; a light positioned on a distal-facing surface of the distal end of the elongated shaft; and a light switch configured to turn the light on and off.

A sheath according to any example described herein may include one or more of the following features: a sheath may further comprise a holder coupled to the proximal end of the shaft, wherein the holder includes a portion that expands radially outward and in a proximal direction from the shaft; the light switch may be positioned on the holder; each of the expandable components may include a balloon; the plurality of expandable components may be arranged around a circumference of the shaft along a same length of the shaft; the sheath may further comprise an inlet protruding from the shaft, the inlet being fluidly coupled to each of the expandable components; the light may extend around a portion of a circumference of the distal end; and the light may include a diffuser coupled to a distal end of a light-transmitting material, and the light-transmitting material may extend along a wall of the shaft from the proximal end to the distal end.

In one example, a method for using a sheath for an imaging device may include inserting at least a portion of a tubular member of the imaging device into a lumen of the sheath; fixing the tubular member relative to the sheath by expanding a retainer positioned in the lumen of the sheath; turning on a light located on a distal-facing surface of the sheath; passing a distal end of the sheath and a distal end of the tubular member of the imaging device through a urethra of a patient and into a bladder of the patient; passing the distal end of the sheath and the distal end of the tubular member into a ureter of the patient; and collapsing the retainer.

A method according to any of the examples described herein may include one or more of the following features or steps: the method may further comprise locating a ureteral orifice of the patient; the method may further comprise, after collapsing the retainer, moving the tubular member relative to the sheath; the retainer may include a plurality of expandable components, and expanding the retainer may include passing fluid into each of the plurality of expandable components; and the method may further comprise inserting the distal end of the tubular member of the imaging device through a funnel-shaped holder prior to inserting the at least a portion of the tubular member of the imaging device into the lumen of the sheath.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

### BRIEF DESCRIPTION OF THE DRAWINGS

The accompanying drawings, which are incorporated in and constitute a part of this specification, illustrate aspects of the present disclosure and together with the description, serve to explain the principles of the disclosure.

FIG. 1 illustrates a sheath with a tubular member of an imaging device within a lumen of the sheath.

FIG. 2 illustrates a longitudinal cross-sectional view of a distal end of a sheath, with a retainer in a collapsed state.

FIG. 3 illustrates a longitudinal cross-sectional view of the retainer of FIG. 2 with the retainer in an expanded state.

FIG. 4 illustrates a light at a distal end of a sheath and a tubular member of an imaging device inside the lumen of the sheath.

FIG. 5 illustrates the components of FIG. 4 after the tubular member has been moved distally relative to the sheath.

FIG. 6 illustrates a sheath and an imaging device during a ureteroscopy procedure.

FIG. 7 illustrates a flow chart of a method for using a sheath with an imaging device.

### DETAILED DESCRIPTION

Examples of the present disclosure may include a sheath for an imaging device. The imaging device may be a ureteroscope or any other device with an imaging system for viewing an interior of a patient. In certain examples, the sheath may include a light that may help to illuminate the patient's anatomy, such as the urethra, bladder, ureter, or kidney. Aspects of the sheath may further include a retainer to fix the sheath relative to a ureteroscope or other imaging device within a lumen of the sheath. The sheath may allow a medical practitioner to perform both a cystoscopy procedure and a ureteroscopy procedure using the same camera, lighting, and other equipment. The related methods disclosed in this application may allow a medical practitioner to eliminate certain steps typically associated with ureteroscopy procedures.

The terms “proximal” and “distal” are used herein to refer to the relative positions of the components of exemplary medical devices. When used herein, “proximal” refers to a position relatively closer to a user of a medical device. In contrast, “distal” refers to a position relatively farther away from the user of a medical device.

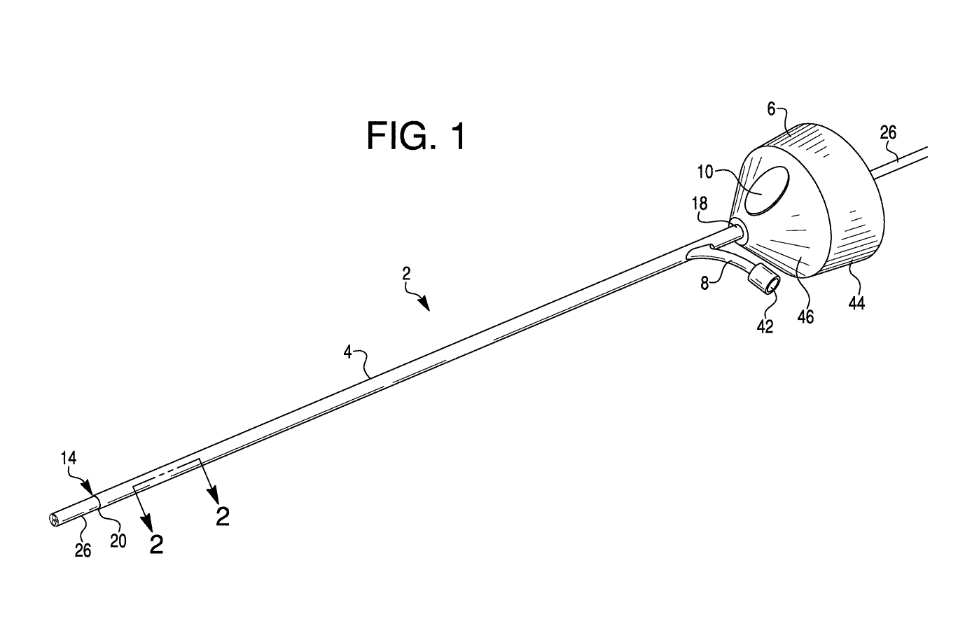

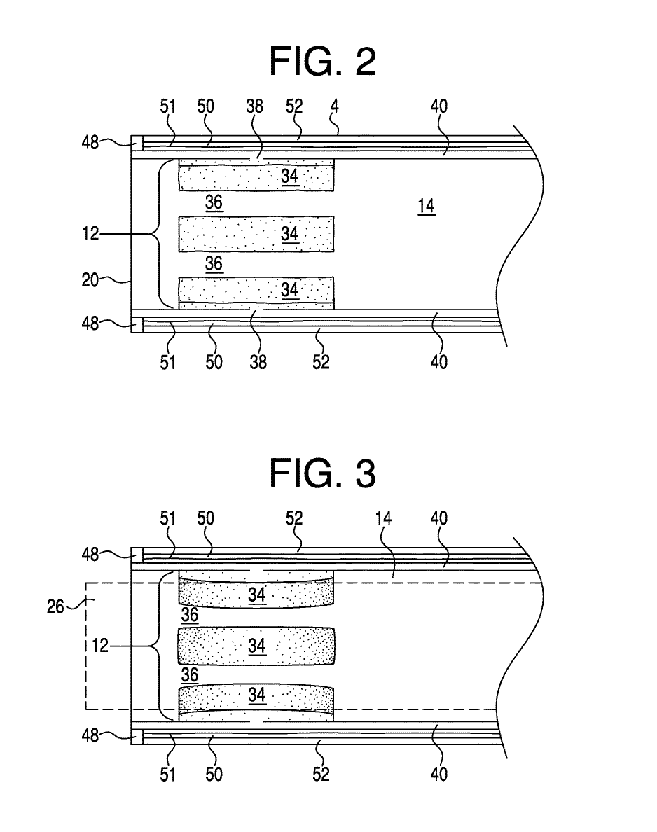

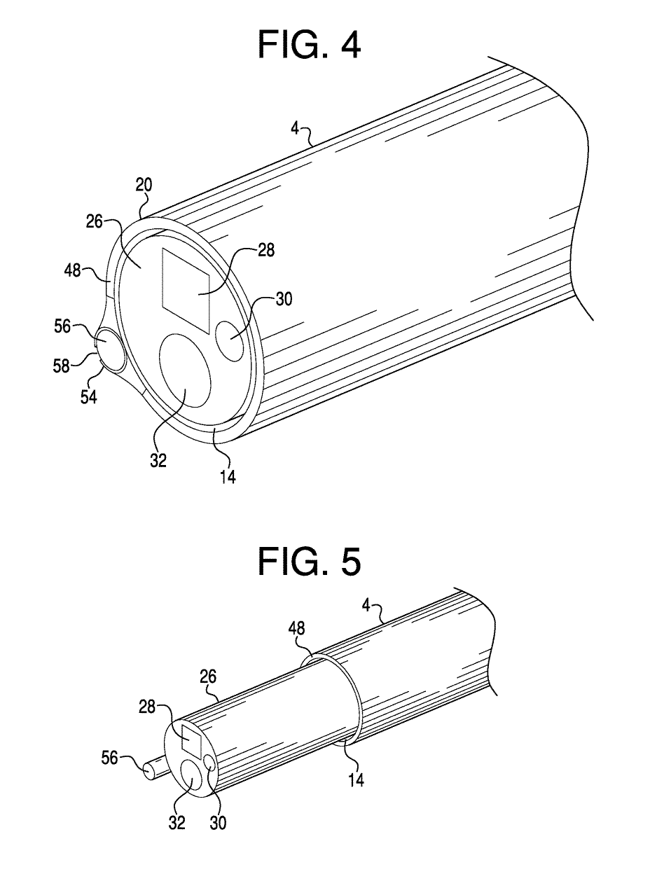

FIG. 1 illustrates a sheath 2 according to one example. Among other components, the sheath may include an elongated shaft 4, an inlet 8 fluidly coupled to a retainer 12 (shown in FIGS. 2 and 3), a holder 6, a light switch 10 to control a light 48 (shown in FIGS. 4 and 5), a main lumen 14, and a side lumen 54 (shown in FIG. 4).

The elongated shaft 4 may extend from a proximal end 18 adjacent the holder 6 to a distal end 20. The shaft 4 may have a length of between 25-55 cm, 28-46 cm, or 28-35 cm. The main portion of the shaft 4, excluding any protrusion from the side lumen 54, may have a diameter of between 1-8 mm, 2-7 mm, 3-6 mm, or 4.5 mm. The outer layer 52 of the shaft 4 (FIGS. 2 and 3) may include any appropriate biocompatible material, such as polymers, urethane, fluorine-polymer blend, or metals (e.g., stainless steel). The shaft 4 may have a rigidity that is sufficient to allow passage of the shaft 4 through a patient's urethra and across the bladder. With sufficient rigidity, the sheath 2 may be used with a tubular member 26 of an imaging device, such as a ureteroscope, to perform a cystoscopy procedure. The cystoscopy procedure may include, for example, passage of an imaging device through the urethra, examination of the bladder, and location of the ureteral orifice connecting the ureter to the bladder.

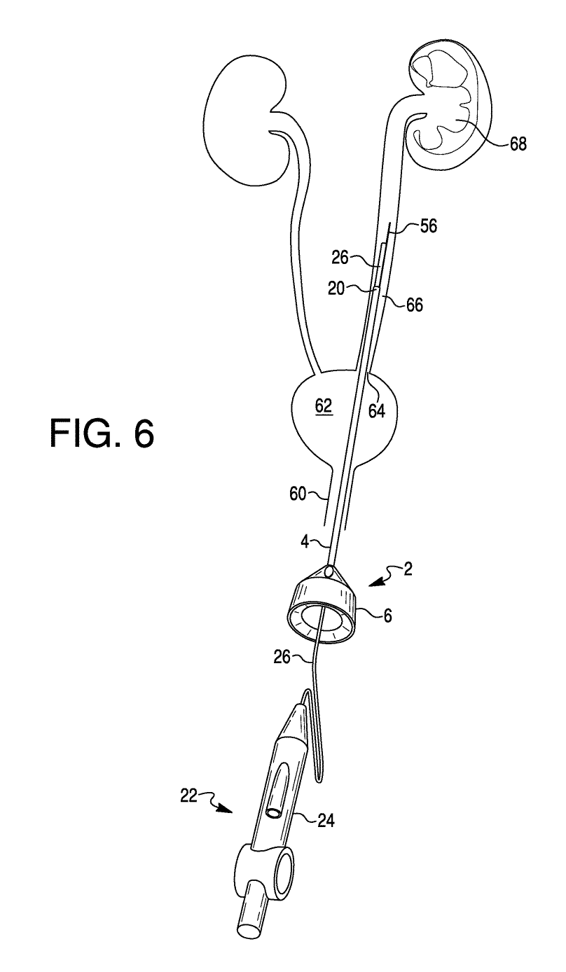

Referring to FIGS. 1 and 6, an imaging device for use with the sheath 2, such as a ureteroscope 22, may include a handle assembly 24 and a tubular member 26. The proximal end of the tubular member 26 may be connected to the handle assembly 24. A distal portion of the tubular member 26 may be positioned within a main lumen 14 of the shaft 4. The ureteroscope 22 may further include a variety of other components necessary for viewing the interior of a body, such as camera and lighting equipment. As shown in FIG. 4, a camera 28 and a light 30 may be located at the distal end of the tubular member 26. Optical fiber cables may be used to transmit light from the proximal end of tubular member 26 to the distal end of tubular member 26. The camera may be coupled to the distal end of tubular member 26, with power and signal wires extending proximal to the ureteroscope handle 24. The tubular member 26 may also include one or more working lumens 32 for tools and irrigation. Working lumen 32 or separate channels in the tubular member 26 may hold the wires and/or fibers connecting the camera 28 and light 30 to a power source, LED, and/or other processing and control equipment, along with mechanical cables to permit maneuverability of the tubular member 26.

Referring to FIGS. 2 and 3, a retainer 12 may be configured to fix the tubular member 26 of the ureteroscope or other imaging device relative to the shaft 4. The retainer 12 may be expandable and collapsible. FIG. 2 illustrates the retainer 12 in a collapsed state. The collapsed state may be achieved by any mechanism that reduces the volume of the retainer 12. For example, liquid may be removed from the retainer 12, as described further below, or the retainer may be actively compressed by another structure. FIG. 3 illustrates the retainer 12 in an expanded state. The expanded state may be achieved by increasing the volume of the retainer 12 relative to the collapsed state by, for example, filling the retainer 12 with fluid, by actively expanding the retainer 12 with another structure, or by allowing the retainer to self-expand. When the retainer 12 is in the collapsed state, the tubular member 26 may be movable relative to the shaft 4. However, when the retainer 12 is in the expanded state, the tubular member 26 may be held in place by retainer 12 and may be fixed relative to the shaft 4.

The retainer may include one or more expandable components 34 lining the main lumen 14 of the shaft 4. FIGS. 2 and 3 illustrate a longitudinal cross-sectional view of the distal end of shaft 4 showing three expandable components 34. The top and bottom expandable components 34 shown in these figures extend away from the viewer (e.g., into the page) and wrap around the lumen 14 towards a middle component 34 positioned on a surface of the lumen 14 farthest from the viewer. In FIG. 2, the expandable components 34 are shown in a collapsed state, and in FIG. 3, the expandable components 34 are shown in an expanded state, protruding towards a central longitudinal axis of the main lumen 14.

In various examples, two, three, four, five, six, or more discrete expandable components 34 may line the circumference of the main lumen 14 of shaft 4 along a portion of the main lumen 14. A cross-section of the shaft 4 taken perpendicular to a longitudinal axis of the shaft may intersect the plurality of expandable components 34 lining the portion of the main lumen 14. In one example, a plurality of expandable components may be arranged around a circumference of the shaft 4 along a same length of the shaft 4.

Each expandable component 34 may include a generally rectangular shape and may be elongated in a longitudinal direction along the length of the shaft 4, with the rectangle being curved against the interior wall of main lumen 14. In other examples, the expandable components may be circular, oval, square, or irregularly shaped. If the retainer 12 includes more than one expandable component 34, as shown in FIGS. 2 and 3, there may be a circumferential space 36 between adjacent components 34. In another example, a single expandable component 34 may extend around all or a portion of the circumference of the main lumen 14.

The expandable components 34 may be located near the distal end 20 of shaft 4. In other examples, the expandable components 34 may be distributed along the length of shaft 4. For example, one or more expandable components 34 may be located near the distal end 20 of the shaft 4, another one or more expandable components 34 may be located near proximal end 18 of the shaft 4, and another one or more expandable components 34 may be located between the distal and proximal expandable components. As yet another example, one or more expandable components 34 may extend along the length of shaft 4, such as from a location adjacent the proximal end 18 to a location adjacent the distal end 20.

Each expandable component 34 may include a balloon that may be expanded by filling the balloon with a fluid (e.g., air, water, saline) and collapsed by removing the fluid from the balloon. Each expandable component 34 may be fluidly connected through an opening 38 (FIG. 2) to an inflation lumen 40 within the wall of the shaft 4. The inflation lumen 40 may be fluidly connected to an inlet 8, shown in FIG. 1. The sheath 2 may include a plurality of separate inflation lumens 40, with each inflation lumen 40 corresponding to one or more expandable components 34. In this example, the expansion and collapse of each expandable component 34 (or each group of expandable components 34) may be individually controlled. Alternatively, the sheath 2 may include a single inflation lumen 40, with each of the expandable components 34 being connected to the same single inflation lumen 40.

The inlet 8 may be an elongated shaft protruding radially outward from a longitudinal axis of the shaft 4 and towards a proximal end of the sheath 2. The inlet 8 may include an inner lumen and a port 42 for connection to a fluid source, such as a container of saline or other fluid. A valve (not shown) may separate the fluid source and the port 42, and a controller (not shown) may regulate fluid flow into and out of the port 42 and the expandable components 34.

Referring to FIG. 1, the sheath 2 may further include a holder 6. The holder may be coupled to the proximal end 18 of the shaft 4. The holder 6 may include a distal portion 46 and a proximal portion 44. From the proximal end 18 of the shaft 4, the inner and/or outer wall of the distal portion 46 of the holder 6 may expand radially outward and in a proximal direction to form a conical shape (e.g., a funnel). The inner and/or outer wall of the proximal portion 44 may include a cylindrical shape or may expand radially outward in a distal-to-proximal direction such that the proximal portion 44 forms a frustum. The funnel-shaped distal portion 46 of the holder 6 may guide the distal end of tubular member 26 or other medical devices into the lumen 14 of shaft 4.

Referring to FIGS. 4 and 5, the distal end 20 of the shaft 4 may include a light 48. The light 48 may be positioned on a distal-facing surface of the shaft 4 and may emit light in a distal direction. Additionally or alternatively, the light 48 may emit light radially outwards (e.g., if the outer surface of the light 48 is exposed on an outer surface, as shown in the example of FIGS. 2 and 3). The light 48 may extend around a portion of the circumference of the distal end 20 of the shaft 4. Alternatively, the light 48 may extend around the full circumference of the distal end 20 of the shaft 4. During procedures within a patient's urinary tract, the light 48 may supplement the light 30 from the tubular member 26 within the shaft 4. The additional light 48 may improve the user's ability to adequately view the patient's anatomy.

The light 48 may be connected to a light source, such as an LED, by a light-transmitting material 51 (shown in the cross sections of FIGS. 2 and 3). The light-transmitting material 51 is illustrated in FIGS. 2 and 3 as optical fibers 51 held within a tubular lumen 50. The light-transmitting material 51 may extend along the length of shaft 4. The light-transmitting material 51 may lie radially outward of the inflation lumen 40 and radially inward of an exterior material 52 (e.g., cladding) of the shaft 4. The light-transmitting material 51 may be any material configured to transmit light from a first location (e.g., an LED) to a second location (e.g., the distal end 20 of the shaft 4). For example, the light-transmitting material 51 may include one or more optical fibers, which may include acrylic (polymethyl methacrylate—PMMA), silicone, polystyrene, or glass.

The light 48 may be defined by the distal end of the light-transmitting material 51 (e.g., the distal ends of optical fibers). Additionally or alternatively, the light 48 may include a diffuser having a different material than the light-transmitting material 50, such as acrylic (PMMA), polystyrene, or glass. The light 48 may be configured to disperse light in a desired pattern. For example, the light 48 may include a curved surface that disperses light in a pattern that extends distally and radially outward from the distal end 20 of the shaft 4.

The proximal end of the light-transmitting material may be connected to any suitable light source, such as an LED. The light source may be located within a wall of the holder 6. The light source may be powered by a battery located within the holder 6. Additionally or alternatively, the light source may be powered by either a battery or a source of electricity located external to the sheath 2.

The holder 6 may include a switch 10 for turning the light source on and off or for adjusting the intensity of the light source to control the light emitted from the light 48 at the distal end 20 of the shaft 4. The switch 10 may include a button that can be pressed by a user to turn the light source off and on or to switch between different intensity settings. Additionally or alternatively, the switch 10 may include a dial that can be rotated or a slide that can be adjusted to control the light source. In other examples, the switch 10 may be separate from the sheath 2 and located, for example, near an external power source.

Referring to FIG. 4, the shaft 4 may include a side lumen 54 for a guidewire 56. The side lumen 54 may be located radially outward of the main lumen 14. The side lumen 54 may be surrounded by material around a portion of its circumference and may include an opening 58 around a portion of its circumference. The material on either side of opening 58 may be flexible to facilitate movement of the guidewire 56 within side lumen 54, as described below. The side lumen 54 may further allow the shaft 4 to be separated from the guidewire 56 without having to remove the tubular member 26 from the patient, as will also be described in greater detail below. In addition, the side lumen 54 may minimize the overall cross-section of the shaft 4, since the main lumen 14 of shaft 4 does not need to accommodate a guidewire. The opening 58 may extend along the shaft 4 from the proximal end 18 to the distal end 20.

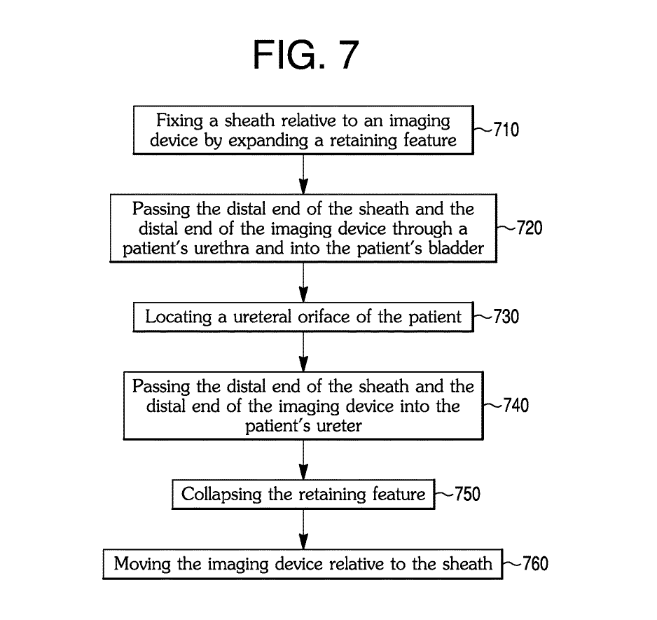

FIGS. 6 and 7 will be referenced, in conjunction with FIGS. 1-5, to describe an exemplary method for using the sheath 2 with an imaging device, such as ureteroscope 22. Initially, the distal end of tubular member 26 of the ureteroscope 22 may be inserted through the holder 6 and into the shaft 4 of sheath 2. The user may urge tubular member 26 through shaft 4 until the distal end of the tubular member 26 aligns with the distal end 20 of shaft 4. The retainer 12 of the shaft 4 may be expanded to fix the tubular member 26 of the ureteroscope 22 relative to the shaft 4 (step 710). The user may use switch 10 to turn on the light 48 of the sheath 4 as a primary or secondary light source, as the light 30 and camera system of the ureteroscope 22 may also optionally be turned on. Using guidance from the camera 28 and lights 30 and 48, the user may pass the distal ends of the shaft 4 and tubular member 26 through the patient's urethra 60 and into the patient's bladder 62 (step 720). The user may further use the camera 28 and lights 30 and 48 to locate a ureteral orifice 64 (step 730). Steps 720 and 730 may be generally referred to as performing a cystoscopy procedure.

After locating the ureteral orifice 64, the user may push guidewire 56 distally into the patient's ureter 66 and possibly into the kidney 68. The flexibility of the material of side lumen 54 may allow the user to more easily move the guidewire 56 relative to the shaft 4 into the ureter 66 and/or kidney 68, as the opening 58 along the side lumen 54 may expand in regions of shaft 4 that are bent. The opening 58 therefore may maintain the flexibility of side lumen 54 and may prevent the guidewire 56 from becoming pinched within its side lumen 54. Once inside the ureter 66, the guidewire 56 may serve as a guide for the shaft 4. If necessary, a balloon may be inserted through the working lumen 32 of the tubular member 26 to dilate the ureteral orifice 64. The balloon may be deflated and the distal ends of the shaft 4 and the tubular member 26 may be inserted into the ureter 66 (step 740). The retainer 12 may be collapsed during any portion of the procedure to allow the tubular member 26 to move relative to the shaft 4 of sheath 2 (step 750). The tubular member 26 may then be moved distally or proximally relative to the shaft 4 of sheath 2 (step 760).

The tubular member 26 may be moved distally relative to the shaft 4 to remove stones lodged in the patient's ureter 66 or kidney 68. Various tools (e.g., lasers, baskets, etc.) may be inserted through the working lumen 32 of the tubular member 26. The tools may be used to break up stones and carry them out of the patient. When the tubular member 26 is extended from the distal end of the first sheath 6, steering control of the tubular member 26 may allow the distal end of the tubular member 26 to navigate tortuous pathways in the ureter 66 or kidney 68 and reach hard-to-access areas to break up and retrieve stones. The tools and/or the tubular member 26 may be removed from the patient one or more times through the shaft 4. The shaft 4 may protect the patient's urethra 60, bladder 62, and ureter 66 from damage that could be caused by passing devices into and out of the patient.

The opening 58 of the side lumen 54 may facilitate removal of the sheath 2 from the patient without having to remove the tubular member 26 from the patient. In this example, the guidewire 56 may enter the shaft 4 distal to the holder 6 and exit the shaft 4 at its distal end 20. Once the tubular member 26 is positioned in the patient, the user may pull the sheath 2 proximally over the tubular member 26 and guidewire 56 until the distal end 20 has been removed from the patient. The shaft 4 can then be separated from the guidewire 56 using the opening 58. If necessary, other tools can then be routed over the guidewire 56 and into the patient.

Use of the sheath 2 as an access sheath in the patient's ureter 66 may eliminate the need to use a separate cystoscope to pass through the urethra 60 and bladder 62, locate the ureteral orifice 64, and place a guidewire into the patient's ureter 66 prior to inserting an access sheath and ureteroscope into the patient's ureter 66. Instead, the sheath 2 is passed through the patient's anatomy once, and all other devices are passed through the sheath 2. The sheath 2 allows the use of a separate cystoscope to be eliminated by providing sufficient rigidity for the tubular member 26 of the ureteroscope 22 to be passed through the urethra 60 and across the bladder 62. Furthermore, the sheath 2 provides additional light to the ureteroscope 22 to aid in viewing the urethra 60, bladder 62, and ureteral orifice 64, since cystoscopes typically provide more light than ureteroscopes. The retainer 12 provides a user-friendly mechanism for allowing the tubular member 26 and the sheath 2 to be fixed and unfixed relative to each other.

Alternatively, the sheath 2 may be placed in the patient's ureter after a different imaging device (e.g., a cystoscope) has been used to place a guidewire into the ureter. For example, a cystoscope may be passed through the patient's urethra and bladder and used to locate the ureteral orifice. A guidewire may then be placed into the patient's ureter. The cystoscope may be removed from the patient. The sheath 2 then may be passed over the guidewire via working lumen 32 or side lumen 54 and into the patient's ureter. A tubular member 26 of a ureteroscope, or other medical devices, may then be passed into and out of the sheath 2 to perform a procedure on the patient.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A sheath for an imaging device, comprising:
a shaft including a proximal end, a distal end, and a lumen extending from the proximal end to the distal end;
a holder coupled to the proximal end of the shaft;
a retainer positioned in the lumen of the shaft, wherein the retainer includes an expanded configuration and a collapsed configuration; and
a light positioned on the distal end of the shaft.

2. The sheath of claim 1, wherein the retainer includes a plurality of expandable components, and each of the expandable components is configured to be transitioned between an expanded configuration and a collapsed configuration.

3. The sheath of claim 2, wherein each of the expandable components includes a balloon.

4. The sheath of claim 2, wherein the plurality of expandable components is arranged around a circumference of an interior wall of the lumen, with a circumferential space between adjacent expandable components, so that a cross-section of the shaft perpendicular to a longitudinal axis of the shaft intersects each of the plurality of expandable components.

5. The sheath of claim 1, wherein the light is positioned on a distal-facing surface of the distal end of the shaft.

6. The sheath of claim 1, wherein the holder includes a light switch configured to turn the light on and off.

7. The sheath of claim 1, further comprising an inlet protruding radially outward from the shaft, the inlet being fluidly coupled to the retainer.

8. A sheath for an imaging device, comprising:
a shaft including a proximal end, a distal end, and a lumen extending from the proximal end to the distal end;
a plurality of expandable components positioned in the lumen, where each of the expandable components is configured to be transitioned between an expanded configuration and a collapsed configuration;
a light positioned on a distal-facing surface of the distal end of the shaft; and
a light switch configured to turn the light on and off.

9. The sheath of claim 8, further comprising a holder coupled to the proximal end of the shaft, wherein the holder includes a portion that expands radially outward and in a proximal direction from the shaft.

10. The sheath of claim 9, wherein the light switch is positioned on the holder.

11. The sheath of claim 8, wherein each of the expandable components includes a balloon.

12. The sheath of claim 11, wherein the plurality of expandable components is arranged around a circumference of the shaft along a same length of the shaft.

13. The sheath of claim 11, further comprising an inlet protruding from the shaft, the inlet being fluidly coupled to each of the expandable components.

14. The sheath of claim 8, wherein the light extends around a portion of a circumference of the distal end.

15. The sheath of claim 8, wherein the light includes a diffuser coupled to a distal end of a light-transmitting material, and wherein the light-transmitting material extends along a wall of the shaft from the proximal end to the distal end.

16. A method for using a sheath for an imaging device, comprising:
inserting at least a portion of a tubular member of the imaging device into a lumen of the sheath;
fixing the tubular member relative to the sheath by expanding a retainer positioned in the lumen of the sheath;
turning on a light located on a distal-facing surface of the sheath;
passing a distal end of the sheath and a distal end of the tubular member of the imaging device through a urethra of a patient and into a bladder of the patient;
passing the distal end of the sheath and the distal end of the tubular member into a ureter of the patient; and
collapsing the retainer.

17. The method of claim 16, further comprising locating a ureteral orifice of the patient.

18. The method of claim 16, further comprising, after collapsing the retainer, moving the tubular member relative to the sheath.

19. The method of claim 16, wherein the retainer includes a plurality of expandable components, and expanding the retainer includes passing fluid into each of the plurality of expandable components.

20. The method of claim 16, further comprising inserting the distal end of the tubular member of the imaging device through a funnel-shaped holder prior to inserting the at least a portion of the tubular member of the imaging device into the lumen of the sheath.
